(19) United States
(12) Patent Application Publication
(10) Pub. No.: US 2002/0099392 A1
(43) Pub. Date: Jul. 25, 2002

(54) Autoanastomosis device and connection technique

(76) Inventors: David H. Mowry, Eden Prairie, MN (US); John M. Schorgl, Eden Prairie, MN (US)

Correspondence Address: MERCHANT & GOULD PC, P.O. BOX 2903, MINNEAPOLIS, MN, 55402-0903, US

(21) Appl. No.: 09/768,930

(22) Filed: Jan. 24, 2001

(51) Int. Cl. A61B 17/08

(52) U.S. Cl. 606/153; 604/8

(57) ABSTRACT

An anastomosis device for securing a biocompatible conduit to a blood vessel. The conduit includes a first end and a second end. A resilient flange is positioned at the second end. The flange is movable between an expanded orientation and a compressed orientation and biased toward the expanded orientation. When the flange is compressed, it is adapted for insertion through an incision in a blood vessel. When the compression is released, the flange returns to its expanded orientation. The first end is adapted to be inserted into and retained within another anatomical structure creating an anastomosis between the blood vessel and the anatomical structure via the conduit. Other anatomical structures specifically include the heart wall of a heart chamber.

### I. BACKGROUND OF THE INVENTION

1. Field of the Invention

This invention pertains to an implant or graft for passing blood flow directly between a blood vessel and another anatomical structure. Other anatomical structures can be a distal portion of the same blood vessel to circumvent an occlusion, another blood vessel or a chamber of the heart. More particularly, this invention pertains to an autoanastomosis device and method.

2. Description of the Prior Art

Anastomosis is the surgical joining of biological tissues, especially the joining of tubular organs to create blood-flow or other body fluid intercommunication between them. Vascular surgery often involves creating an anastomosis between blood vessels or between a blood vessel and a vascular graft to create or restore a blood flow path to essential tissues. Coronary artery bypass surgery (CABS) is a surgical procedure to restore blood flow to ischemic heart muscle whose blood supply has been compromised by occlusion or stenosis of one or more of the coronary arteries.

One method for performing CABS involves harvesting a saphenous vein or other venous or arterial conduit from elsewhere in the body, or using an artificial conduit, such as one made of expanded polytetrafluoroethylene (ePTFE) tubing, and connecting this conduit as a bypass graft from a viable artery or a chamber of the heart to the coronary artery downstream of the blockage or narrowing. In the first case involving the use of a viable artery, the bypass graft is typically attached to the native arteries by an end-to-side anastomosis at both the proximal and distal ends of the graft—the proximal end being the source of the blood and the distal end being the destination of the blood. In the second technique using a chamber of the heart, an end-to-side anastomosis can be made at the distal end of the graft. When performing and “end-to-side” anastomosis, the end of the graft/conduit connected to the native artery is typically aligned along an axis that is generally perpendicular relative to the axis of the artery.

At present, most vascular anastomosis are performed by conventional hand suturing. Suturing the anastomosis is time-consuming and difficult, requiring much skill and practice on the part of the surgeon. During CABS, it is important to complete the anastomosis procedure quickly and efficiently to reduce the risk of complications associated with the procedure.

When the objective of CABS involves creating anastomosis between a chamber of the heart and a coronary vessel, the graft can have special compression-resistant characteristics. U.S. Pat. No. 5,944,019 issued Aug. 31, 1999, which is hereby incorporated by reference, teaches an implant for defining a blood flow conduit directly from a chamber of the heart to a lumen of a coronary vessel. An embodiment disclosed in the aforementioned patent teaches an L-shaped implant in the form of a rigid conduit having one leg sized to be received within a lumen of a coronary artery and a second leg sized to pass through the myocardium and extend into the left ventricle of the heart. As disclosed in the above-referenced patent, the conduit is rigid and remains open for blood flow to pass through the conduit during both systole and diastole. The conduit penetrates into the left ventricle in order to prevent tissue growth and occlusions over an opening of the conduit.

U.S. Pat. No. 5,984,956 issued Nov. 16, 1999 teaches an implant with an enhanced fixation structure. The enhanced fixation structure includes a fabric surrounding at least a portion of the conduit to facilitate tissue growth on the exterior of the implant. U.S. Pat. No. 6,029,672 issued Feb. 29, 2000 teaches procedures and tools for placing a conduit.

Implants such as those shown in the aforementioned patents include a portion to be connected to a coronary vessel (distal end) and a portion to be placed within the myocardium (proximal end). Most of the implants disclosed in the above-mentioned patents are rigid structures. Being rigid, the implants are restricted in use. For example, an occluded site may not be positioned on the heart in close proximity to a heart chamber containing oxygenated blood. To access such a site with a rigid, titanium implant, a relatively long implant must be used. A long implant results in a long pathway in which blood will be in contact with the material of the implant. With non-biological materials, such as titanium, a long residence time of blood against such materials increases the probability of thrombus. The risk can be reduced with anti-thrombotic coatings. Moreover, a rigid implant can be difficult to place while achieving desired alignment of the implant with the vessel. A flexible implant will enhance placement of the implant. U.S. Pat. No. 5,944,019 shows a flexible implant in FIG. 22 of the '019 patent by showing a cylindrical rigid member in the heart wall and a T-shaped rigid member in the coronary artery. The cylindrical and T-shaped rigid members are joined by flexible conduit. Unfortunately, flexible materials tend to be non-biostable and trombogenic and may collapse due to contraction of the heart during systole. PCT/US99/01012 shows a flexible transmyocardial conduit in the form of a cylindrical rigid member in the heart wall and a natural vessel (artery or vein segment) connecting the rigid member to an occluded artery. PCT/US99/00593 (International Publication No. WO99/38459) also shows a flexible conduit. PCT/US97/14801 (International Publication No. WO 98/08456) shows (in FIG. 8c) a transmyocardial stent with a covering of expanded polytetrafluoroethylene.

The above-referenced inventions clearly demonstrate the need for an implant that is partially flexible, yet rigid enough to withstand the contraction forces of the heart. Certain aspects of the present invention satisfy that need and also incorporate a novel device to create an end-to-side auto-anastomosis with a coronary or other blood vessel.

U.S. Pat. No. 4,214,587, issued Jul. 29, 1980, teaches the use of a plurality of barbs to create an end-to-end anastomosis. The obvious limitation of this device is that it is not suitable for end-to-side anastomosis. As discussed above, end-to-side anastomosis is the primary objective in CABS.

U.S. Pat. No. 6,171,321, issued Jan. 9, 2001, teaches the use of a vascular anastomosis staple device to perform an end-to-side anastomosis between a graft vessel and the wall of a target vessel. However, using staples as taught by this invention requires the surgeon to perform complex manual manipulations or use special tools to insert and then deform the staples to create an end-to-side anastomosis.

To solve the above-identified problems as well as other problems, it is desirable to minimize complex and difficult manual manipulations to create an end-to-side anastomosis. An important aspect of the present invention relates to a device for efficiently creating a side-to-end anastomosis.

### III. SUMMARY OF THE INVENTION

According to a preferred embodiment of the present invention, an anastomosis device is disclosed for securing a biocompatible conduit to a blood vessel. The conduit includes a first end and a second end. A flange is positioned at the second end. The flange is movable between an expanded orientation and a compressed orientation and has a resilient construction that biases the flange toward the expanded orientation. The flange projects radially outward from the conduit and extends about a circumference of the conduit when in the expanded orientation. When the flange is in its compressed orientation, it is adapted for insertion through an incision cut within a wall of a blood vessel. After the flange has been inserted into the blood vessel through the incision, the flange is released from compression and returns to its expanded orientation. To complete the anastomosis, the flange can be secured to the blood vessel using a plurality of anchoring teeth. Alternatively, for some applications, the flange is secured in place within the vessel by the natural fluid pressure within the vessel.

### IV. BRIEF DESCRIPTION OF THE DRAWINGS

FIG. 1 is a side sectional view of an implant according to the present invention;

FIG. 2 is a side sectional view of an implant according to the present invention shown in place in a human heart wall with the implant establishing a direct blood flow path from a heart chamber to a coronary vessel;

FIG. 3 is a perspective view of a novel attachment member for attachment to a vessel in lieu of a conventional anastomosis;

FIG. 4 is a longitudinal cross-sectional view of the anastomosis device of FIG. 3 with the device shown in an expanded orientation;

FIG. 5 is a longitudinal cross-sectional view of the anastomosis device of FIG. 3 with the device shown in a compressed orientation;

FIG. 6 is an end view of the anastomosis device of FIG. 3;

FIG. 7 is a cross-sectional view of an alternative anastomosis device shown in an expanded orientation;

FIG. 8 is a cross-sectional view of the anastomosis device of FIG. 7 shown in a compressed orientation;

FIG. 9 shows a resilient ring used in the device of FIGS. 7 and 8; and

FIG. 10 is a side sectional view of the anastomosis device of FIG. 7 showing anchoring teeth of the device embedded in a vessel wall.

### V. DETAILED DESCRIPTION

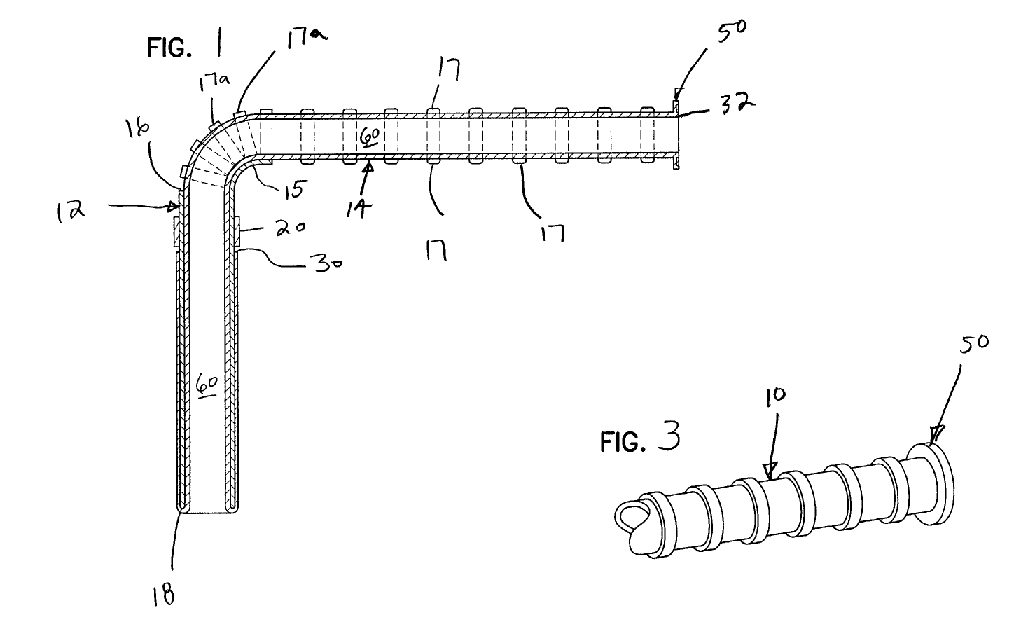

With initial reference to FIGS. 1-3, an implant 10 is shown including a composite of a hollow, rigid cylindrical conduit 12 and a flexible conduit 14. The conduit 12 may be formed of any suitable material. In a preferred embodiment conduit 12 is formed of low density polyethylene (“LDPE”). The conduit 12 preferably has a rigid construction. The term “rigid” will be understood to mean that the conduit is sufficiently rigid to withstand contraction forces of the myocardium and hold open a path through the myocardium during both systole and diastole.

The conduit 12 is sized to extend through the myocardium MYO of the human heart to project into the interior of a heart chamber HC (preferably, the left ventricle) by a distance of about 5 mm. In certain embodiments, the conduit 12 has a length in the range of 20-35 millimeters. The conduit 12 extends from a first (or upper) end 16 to a second (or lower) end 18 (FIG. 1).

As discussed more fully in the afore-mentioned U.S. Pat. No. 5,984,956, the conduit 12 may be provided with tissue-growth inducing material 20 adjacent the upper end 16 to immobilize the conduit 12 within the myocardium MYO. The material 20 surrounds the exterior of the conduit 12 and may be a polyester woven sleeve or sintered metal to define pores into which tissue growth from the myocardium MYO may occur.

The flexible conduit 14 has first and second ends 30, 32 (FIG. 1). In one non-limiting embodiment, the conduit 14 has an inner diameter in the range of 2.5-3.5 millimeters. The first end 30 of the flexible conduit 14 is inserted through the interior of the conduit 12. The first end 30 is wrapped around the lower end 18 of the conduit 12 such that the first end 30 of the graft 14 covers the exterior of the conduit 12 adjacent the lower end 18 of the conduit 12. The first end 30 terminates spaced from the upper end 16 to expose the tissue-growth inducing material 20.

The first end 30 of the flexible conduit 14 can be secured to the rigid conduit 12 by heat bonding along all surfaces of opposing material of the rigid conduit 12 and the flexible conduit 14. At elevated temperatures, the material of the rigid conduit 12 flows into the micro-pores of the material of the flexible conduit 14. The rigid material has a lower melting point than the flexible material.

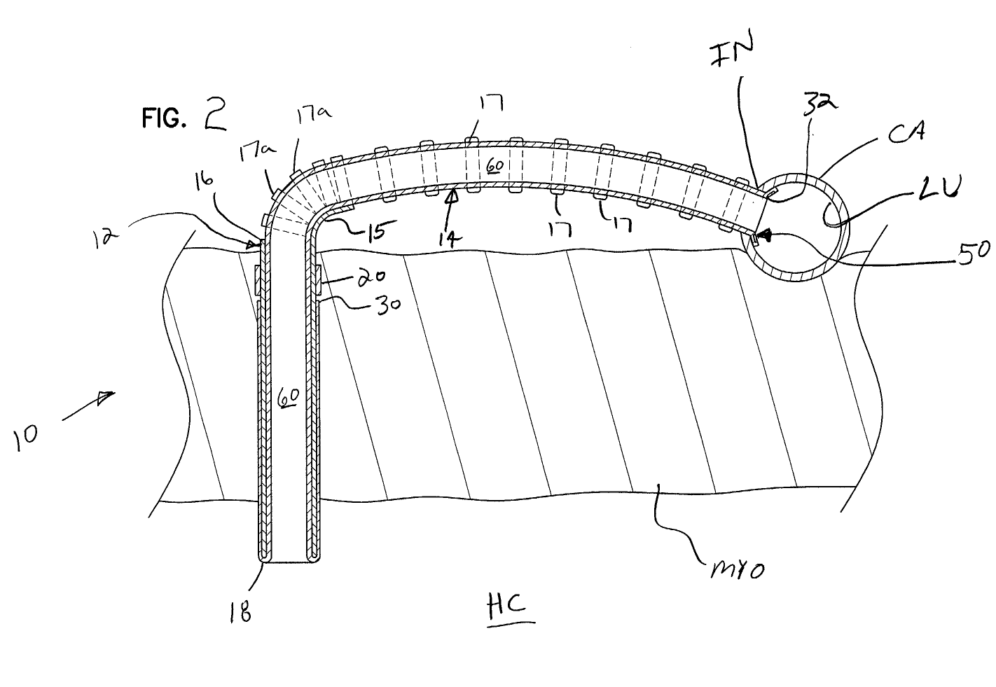

The rigid conduit 12 and attached flexible conduit 14 are preferably placed in the myocardium MYO with the lower end 18 protruding into the left ventricle HC. The implant 10 defines an open blood flow path 60 that provides blood flow communication directly between the left ventricle HC and the lumen LU of a coronary vessel CA lying at an exterior of the heart wall MYO (see FIG. 2). To bypass an obstruction in a coronary artery, the end 32 of the flexible conduit is attached to the artery CA. For example, the end 32 may be anastomosed to the artery CA in an end-to-side anastomosis with an anastomosis device 50. The end 32 is secured to the artery CA distal (i.e., downstream from) to the obstruction.

With the above-described embodiment, the implant 10 permits revascularization from the left ventricle HC to a coronary vessel such as a coronary artery CA (or a coronary vein in the event of a retrograde profusion procedure). The use of an elongated, flexible conduit 14 permits revascularization where the vessel CA is not necessarily in overlying relation to the chamber HC. For example, the implant 10 permits direct blood flow between the left ventricle HC and a vessel CA overlying the right ventricle (not shown). The use of a PTFE flexible conduit 14 results in blood flowing through path 60 being exposed only to PTFE which is a material already used as a synthetic vessel with proven blood and tissue compatibility thereby reducing risk of thrombosis and encouraging endotheliazation. As shown in FIG. 1, the graft 14 is wrapped around the conduit 12 so that no portion of the rigid conduit 12 is in contact with blood within the left ventricle HC.

An interior radius 15 (FIG. 1) is provided on a side of the rigid conduit 12 at end 16. The radius 15 provides support for the flexible conduit 14 and pre-forms the flexible conduit at a preferred 90° bend (a bend of differing degree or no bend could be used).

A plurality of discrete rigid rings 17 are provided along the length of the flexible conduit that is not co-extensive with the rigid conduit. Preferably, the rings are LDPE each having an interior surface heat bonded to an exterior surface of the flexible conduit 14. At the radius 15, LDPE rings 17a are integrally formed with the radius 15 with the cross-sectional planes of the rings 17a set at a fixed angle of separation (e.g., about 20 degrees) to support the flexible conduit throughout the 90 degree bend. Again, an interior surface of rings 1 7a is heat bonded to an exterior surface of the flexible conduit. The rings 17, 17a provide crush resistance. Between the rings 17, 17a, the flexible conduit may flex inwardly and outwardly to better simulate the natural compliance of a natural blood vessel. By way of a further non-limiting example, the discrete rings 17 could be replaced with a continuous helix.

With the foregoing design, an implant of accepted implant material (e.g., LDPE, ePTFE or other bio-compatible material) is formed with blood only exposed to the higher blood compatibility material. The constantly open geometry permits a smaller internal diameter of the ePTFE than previously attainable with conventional grafts.

FIGS. 3-9 illustrate an invention for attaching a conduit to a vessel in other than a traditional end-to-side anastomosis while permitting blood to flow from the conduit and in opposite directions with a vessel. The embodiment of the invention is illustrated with respect to use with the conduit 10 of FIG. 1 but may be used with any suitable conduit or graft material. Further, the anastomosis device is not limited to performing a heart to vessel type anastomosis. For example, the anastomosis device can be used to provide a vessel to vessel type anastomosis.

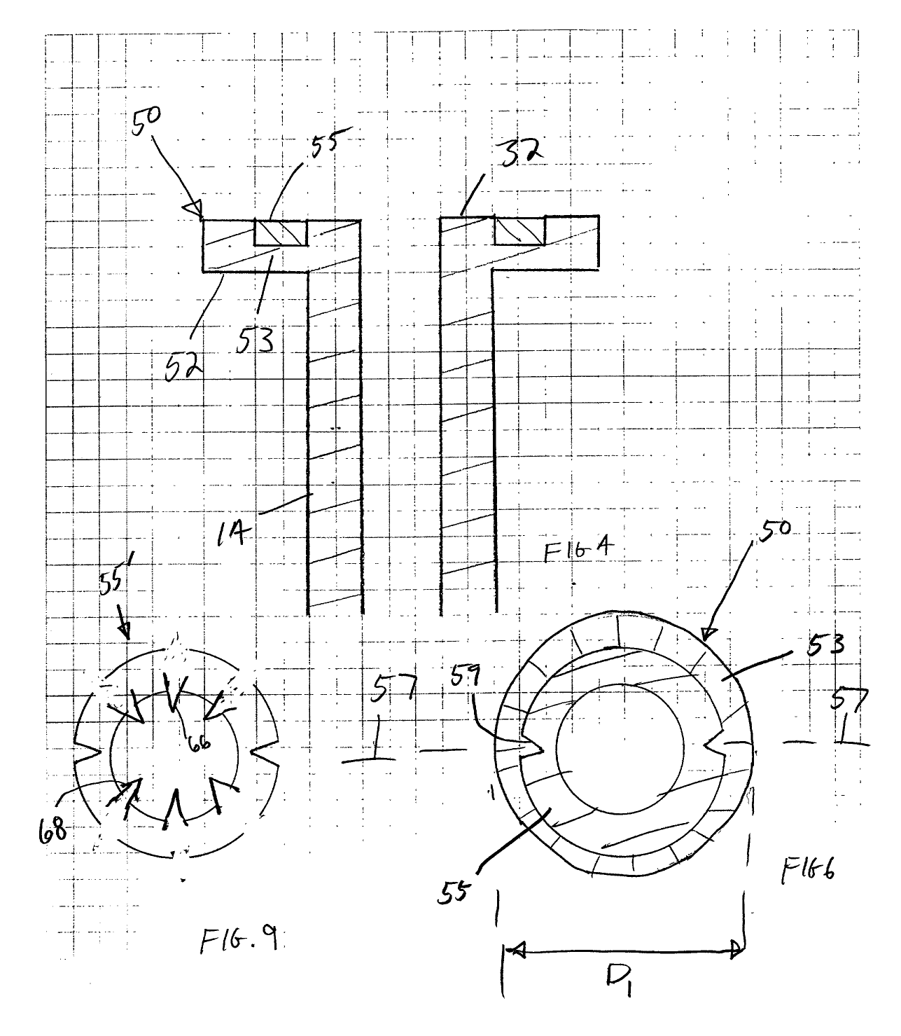

Referring to FIG. 4, the anastomosis device 50 includes a flange 52 positioned at the second end 32 of the flexible conduit 14. The flange 52 includes a main body 53 that is integrally formed (i.e., unitarily or monolithically formed as a common, seamless piece) with the body of the flexible conduit 14. For example, the main body 53 of the flange 52 and the conduit 14 can be integrally formed of ePTFE. Alternatively, the flange 52 can be a separate piece that is bonded or otherwise secured to the second end 32 of the flexible conduit 14.

The flange 52 is movable between an expanded orientation (shown in FIG. 4) and a compressed orientation (shown in FIG. 5). In the expanded orientation, the flange 52 projects radially outwardly from the flexible conduit 14 and has an enlarged shape or perimeter. For example, as shown in FIG. 3, the flange 52 circumferentially surrounds (i.e., concentrically surrounds) the conduit 14 and has a generally circular shape. Preferably the outer diameter of the flange 52 is larger than the outer diameter of the flexible conduit 14. In one non-limiting embodiment, the flange has an outer diameter in the range of 3-5.5 millimeters. In another embodiment, the flange has an outer diameter in the range of 10% to 100% larger than the outer diameter of the flexible conduit 14. While a circular shape is preferred, other shapes such as elliptical shapes, oblong shapes and obround shapes could also be used. Further, for certain applications it may be desirable to use a non-round shape (e.g., square).

The flange 52 preferably includes a biasing structure for resiliently biasing (i.e., in a spring-like manner) the flange 52 toward the expanded orientation. For example, the resilient structure can be provided by the inherent properties of the materials selected to make the main body 53 of the flange 52. Alternatively, a separate resilient structure can be connected to (i.e., embedded in, bonded to, fastened to, or otherwise secured to) the main body of the flange 52. For example, FIG. 4 shows a resilient structure in the form of resilient ring 55 embedded in the main body 53 of the flange 52. The ring 55 is preferably made of an elastic or superelastic material. In one embodiment, the ring 55 is made of a metal that exhibits elastic or superelastic characteristics such as a nickel titanium alloy.

As shown in FIG. 5, the flange 52 is moved to the compressed orientation by folding the flange 52 upwardly about fold line 57 (best shown in FIG. 6). In alternative embodiments, the flange could also be folded downwardly about fold line 57. The fold line 57 can be defined by a hinge 59 (e.g., regions of reduced thickness) provided on the ring 55. When moved to the compressed orientation, the flange 52 is folded about fold line 57 into two generally semi-circular halves. With the flange 52 oriented in the folded configuration, the outer diameter D₁ (labeled in FIG. 6) in a direction taken along fold line 57 is equal to the outer diameter of the expanded flange 52. However, when in the compressed orientation, the outer diameter D₂ (labeled in FIG. 5) in a direction that is transverse relative to the fold line is substantially reduced as compared to the outer diameter of the expanded flange 52. By reducing the diameter in at least one direction, the flange 52 can be passed through a vessel incision IN (shown in FIG. 2) having a size approximately the same as the outer diameter of the flexible conduit 14. This can be accomplished by manipulating the conduit 14 relative to the vessel such that a first end of the fold line is initially inserted through the opening, and the opposite end of the fold line is subsequently passed through the incision IN.

During the insertion process, the flange 52 is preferably held in the compressed orientation by a retaining tool (not shown) such as a forceps or a retractable sheath or collar. If a cylindrical sheath is used to hold the flange 52 in the compressed orientation, the flange 52 can be folded or otherwise collapsed into a generally conical configuration. If a forceps is used, the physician uses the forceps to manually hold the flange 52 in the folded orientation until after insertion in the vessel. Once the flange 52 has been inserted within the vessel, the flange can be released from the retaining tool thereby allowing the flange 52 to self-expand to the expanded orientation within the vessel (see FIG. 2). Once expanded, blood pressure within the vessel preferably secures the flange 52 against the wall of the vessel thereby limiting movement of the flange and eliminating the need for sutures. However, for some applications, sutures or bio-glue can also be used to secure the flange 52 to the vessel.

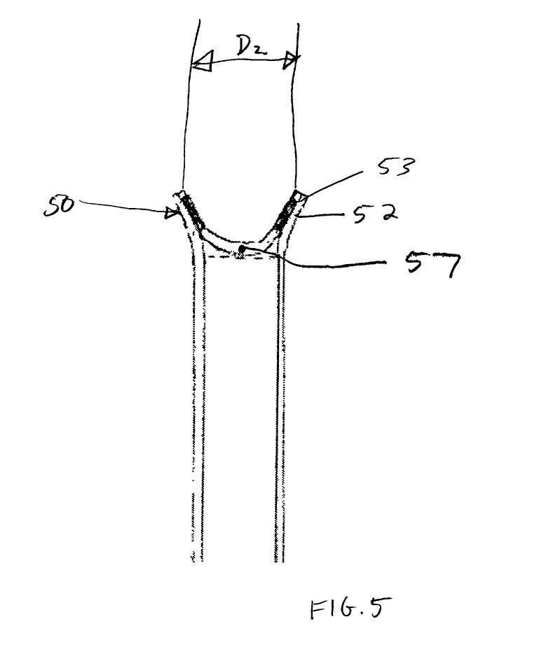

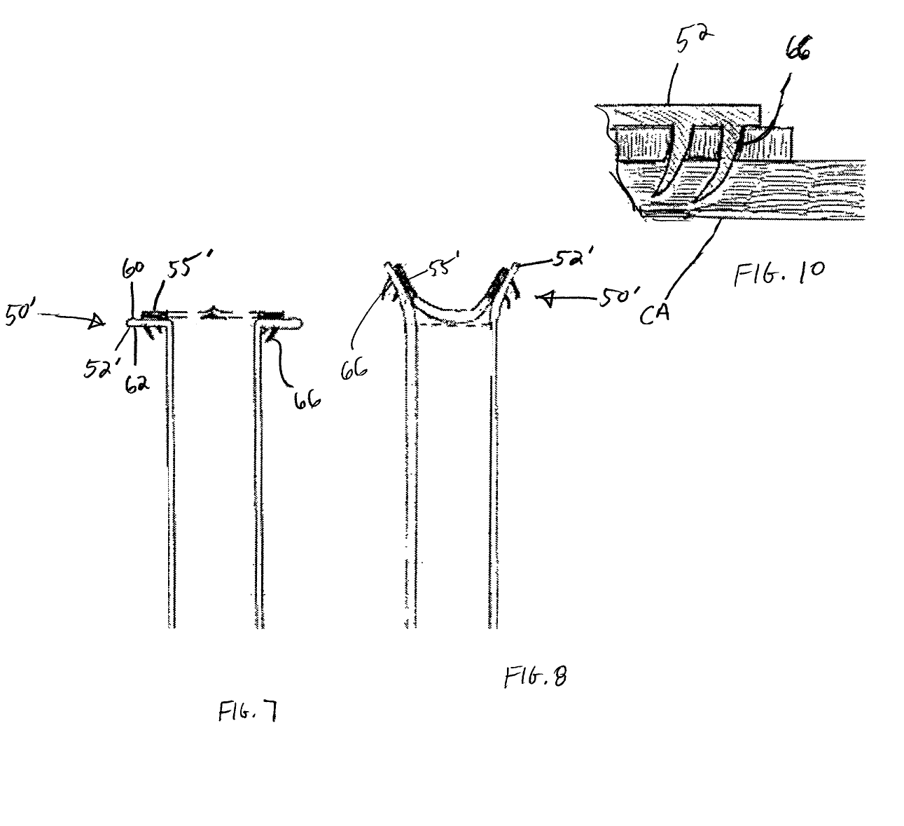

FIGS. 7-9 show another anastomosis device 50′ constructed in accordance with the principles of the present invention. The anastomosis device 50′ includes a flange 52′ having a top side 60 positioned opposite from a bottom side 62. A resilient ring 55′ is connected to the top side 60 of the flange 52′. The ring 55′ is secured to the flange by teeth 66 that extend from the top side 60 through the bottom side 62. As shown in FIG. 9, the teeth 66 can include one or more optional barbs 68.

To attach the device 50′ to a vessel, an incision IN is formed in a blood vessel. Next, the flange 52′ is compressed. After compression, the flange end is inserted into the lumen of the vessel through the incision. After the flange 52′ has been inserted into the lumen, the flange 52′ is released from compression thereby allowing the flange 52′ to self expand to the expanded orientation. Upon expansion of the flange 52′, the teeth 66 projecting beyond the bottom side 62 of the flange 52′ embed within the inner wall of the vessel CA to create an auto-anastomosis (see FIG. 10). The device 50′ can then be manipulated to ensure that the teeth 66 are fully embedded in the inner wall of the vessel. The barbs 68 of the teeth 66 allow the teeth 66 to penetrate the inner wall of the vessel, but prevent the teeth from withdrawing once in place.

Having disclosed the present invention in a preferred embodiment, it will be appreciated that modifications and equivalents may occur to one of ordinary skill in the art having the benefits of the teachings of the present invention. It is intended that such modifications shall be included within the scope of the claims are appended hereto.

What is claimed is:

1. An anastomosis device comprising:
a biocompatible conduit having a first end and a second end;
a flange positioned at the second end of the conduit, the flange being movable between an expanded orientation and a compressed orientation, the flange having a resilient construction that biases the flange toward the expanded orientation;
the flange being adapted for insertion through an incision in a blood vessel when in the compressed orientation; and
the flange projecting radially outward from the conduit and extending about a circumference of the conduit when in the expanded orientation, wherein the flange is adapted to be secured to the blood vessel when in the expanded orientation.

2. The device of claim 1 wherein:
the conduit is formed of expanded polytetrafluoroethylene (ePTFE).

3. The device of claim 1 wherein:
the flange includes a first portion, a second portion, a topside and an underside, the first portion being integrally formed with the conduit, and the second portion includes a ring of resilient metal embedded in the first portion.

4. The device of claim 1 wherein:
the first end of the conduit is formed of a rigid material to withstand contraction forces of the myocardium and hold open a path through the myocardium during both systole and diastole.

5. The device of claim 3 wherein:
the ring of resilient metal includes a hinge for allowing the flange to be folded to the compressed orientation.

6. The device of claim 3 wherein:
said ring of resilient metal is formed from an alloy of Nickel and Titanium.

7. The device of claim 3 wherein:
said ring of resilient metal contains a plurality of anchoring teeth that protrude from the flange for embedding in the vessel to provide a secure connection therein between, said teeth project from the flange and extend toward the first end of the conduit.

8. The device of claim 4 wherein:
said rigid material is low-density polyethylene encapsulated in expanded polytetrafluoroethylene.

9. The device of claim 6 wherein:
said ring of Nickel-Titanium metal alloy contains a plurality of anchoring teeth that protrude from the flange and beyond a plane formed by the underside of the flange in a manner generally perpendicular to the plane formed by the flange.

10. The device of claim 7 wherein:
the plurality of anchoring teeth secure the flange to an inner wall of the blood vessel.

11. The device of claim 7 wherein:
said plurality of anchoring teeth contains a plurality of barbs that protrude from the anchoring teeth.

12. The device of claims 1 wherein:
the device is used in a coronary artery bypass procedure at a coronary vessel disposed lying at an exterior of a heart wall.

13. A method for providing a connection with a blood vessel by using a device having a conduit including first and second ends and a compressible flange located at the second end, wherein the method comprises:
a) incising the vessel to provide an incision;
b) compressing the flange;
c) inserting the compressed flange through the incision;
d) expanding the flange from the compressed orientation to an expanded orientation after the flange has been inserted through the incision; and
e) securing the expanded flange to the vessel with the flange positioned within a lumen of the vessel and the conduit extending outwards through the incision made in the vessel.

14. The method of claim 13 wherein:
the first end of the conduit is retained within a heart wall of a heart chamber containing oxygenated blood, with the conduit in blood-flow communication with blood contained within the chamber.

15. The method of claim 13 wherein:
said compression is accomplished by manual compression of the flange using a forceps.

16. The method of claim 13 wherein:
said compression is accomplished by a movable collar that encircles the flange.

17. The method of claim 13 wherein:
said securing is accomplished by a plurality of anchoring teeth that attach the flange to the vessel.

18. The method of claim 13 wherein:
said securing is accomplished by a plurality of anchoring barbed teeth protruding from the flange that attach the flange to the vessel creating an autoanastomosis.
